(19) United States
(12) Patent Application Publication
(10) Pub. No.: US 2022/0143123 A1
(43) Pub. Date: May 12, 2022

(54) Prevention of Pathological Coagulation in COVID-19 and other Inflammatory Conditions

(71) Applicant: Therapeutic Solutions International, Inc., Oceanside, CA (US)

(72) Inventors: Thomas E. Ichim, Oceanside, CA (US); James Veltmeyer, Oceanside, CA (US); Timothy G. Dixon, Oceanside, CA (US)

(21) Appl. No.: 17/473,741

(22) Filed: Sep. 13, 2021

Related U.S. Application Data
(60) Provisional application No. 63/050,886, filed on Jul. 13, 2020.

(51) Int. Cl. A61K 36/71; A61K 36/45; A61K 36/31; A61K 36/82; A61K 31/353; A61K 31/09; A61K 31/122; A61K 31/26; A61P 37/06

(52) U.S. Cl. CPC A61K 36/71; A61K 36/45; A61K 36/31; A61K 36/82; A61P 37/06; A61K 31/09; A61K 31/122; A61K 31/26; A61K 31/353

(57) ABSTRACT

The invention is directed to the utilization of pterostilbene, and/or nigella sativa extract, and/or sulforaphane, and/or Epigallocatechin gallate (EGCG) alone or in combination, for the prevention of pathological coagulation. In on embodiment a composition containing all four ingredients is administered to a patient at risk of hypercoagulation in order to prevent aberrant expression of pro-coagulation molecules and/or induce expression of molecules known to suppress coagulation. In one embodiment the invention teaches administration of pterostilbene, thymoquinone, sulforaphane, and EGCG as a means of decreasing expression of tissue factor.

### CROSS-REFERENCE TO RELATED APPLICATIONS

This application claims priority to U.S. Provisional Application No. 63/050,886, filed Jul. 13, 2020, which is incorporated herein by reference in its entirety.

### FIELD OF THE INVENTION

The invention pertains to the area of inflammation, more particularly, the invention pertains to inhibition of effects of inflammation on the coagulation system, more particularly, the invention teaches means of suppressing inflammation induced expression of coagulation promoting factors.

### BACKGROUND OF THE INVENTION

The highly contagious coronavirus, SARS-CoV-2 (previously known as 2019-nCoV), is spreading rapidly around the world, causing a sharp rise of a pneumonia-like disease termed Coronavirus Disease 2019 (COVID-19) [1, 2]. COVID-19 presents with a high mortality rate, estimated at 3.4% by the World Health Organization [3]. The rapid spread of the virus (estimated reproductive number RO 2.2-3.6 [4, 5] is causing a significant surge of patients requiring intensive care. More than 1 out of 4 hospitalized COVID-19 patients have required admission to an Intensive Care Unit (ICU) for respiratory support, and a large proportion of these ICU-COVID-19 patients, between 17% and 46%, have died [6-10].

A common observation among patients with severe COVID-19 infection is an inflammatory response localized to the lower respiratory tract [11-13]. This inflammation, associated with dyspnea and hypoxemia, in some cases evolves into excessive immune response with cytokine storm, determining progression to Acute Lung Injury (ALI), Acute Respiratory Distress Syndrome (ARDS), organ failure, and death [2, 10]. Draconian measures have been put in place in an attempt to curtail the impact of the COVID-19 epidemic on population health and healthcare systems. WHO has now classified COVID-19 a pandemic [3].

At the present time, there is neither a vaccine nor specific antiviral treatments for seriously ill patients infected with COVID-19. Crucially, no options are available for those patients with rapidly progressing ARDS evolving to organ failure. Although supportive care is provided whenever possible, including mechanical ventilation and support of vital organ functions, it is insufficient in most severe cases. Therefore, there is an urgent need for novel therapies that can dampen the excessive inflammatory response in the lungs, associated with the immunopathological cytokine storm, and accelerate the regeneration of functional lung tissue in COVID-19 patients.

### SUMMARY

Preferred embodiments are directed to methods of reducing inflammation associated hypercoagulation states comprising administration of a therapeutic combination comprising of: a) Green Tea and/or extract thereof; b) Blueberry and/or extract thereof; c) Nigella Sativa and/or extract thereof; and d) broccoli and/or extract thereof.

Preferred embodiments are directed to methods wherein said green tea extract is epigallocatechin-3-gallate or an analogue thereof.

Preferred embodiments are directed to methods wherein said blueberry extract is pterostilbene or an analogue thereof.

Preferred embodiments are directed to methods wherein said Nigella Sativa extract is thymoquinone or an analogue thereof.

Preferred embodiments are directed to methods wherein said broccoli extract is sulforaphane or an analogue thereof.

Preferred embodiments are directed to methods wherein said therapeutic combination is administered at a dosage and frequency sufficient to inhibit tissue factor expression.

Preferred embodiments are directed to methods wherein inhibition of tissue factor expression occurs when tissue factor is expressed at a basal level.

Preferred embodiments are directed to methods wherein inhibition of tissue factor expression occurs when tissue factor is expression is induced.

Preferred embodiments are directed to methods wherein said tissue factor expression is induced by viral infection.

Preferred embodiments are directed to methods wherein viral infection directly induces expression of tissue factor.

Preferred embodiments are directed to methods wherein viral infection induces expression of cytokines which induce expression of tissue factor.

Preferred embodiments are directed to methods wherein tissue factor expression is inhibited on endothelial cells.

Preferred embodiments are directed to methods wherein tissue factor expression is inhibited on pericytes.

Preferred embodiments are directed to methods wherein said tissue factor inducing cytokine is TNF-alpha.

Preferred embodiments are directed to methods wherein said tissue factor inducing cytokine is IL-6.

Preferred embodiments are directed to methods wherein said tissue factor inducing cytokine is IL-1.

Preferred embodiments are directed to methods wherein said tissue factor inducing cytokine is IL-8.

Preferred embodiments are directed to methods wherein said viral life cycle comprises of: a) entry; b) propagation; and c) budding.

Preferred embodiments are directed to methods wherein said therapeutic mixture decreases hypercoagulability state by inducing upregulated expression of thrombomodulin.

Preferred embodiments are directed to methods wherein said therapeutic mixture decreases hypercoagulability state by inducing upregulated expression of anti-thrombin III.

Preferred embodiments are directed to methods wherein said therapeutic mixture decreases hypercoagulability state by inducing upregulated expression of Protein C.

Preferred embodiments are directed to methods wherein said therapeutic mixture decreases hypercoagulability state by inducing upregulated expression of CD39.

Preferred embodiments are directed to methods wherein said composition reduces propensity of endothelium for hypercoagulation by reducing endothelial injury.

Preferred embodiments are directed to methods wherein said reduction of endothelial injury is suppression of endothelial adhesion molecules associated with inflammation.

Preferred embodiments are directed to methods wherein said adhesion molecule associated with inflammation is E-Selectin.

Preferred embodiments are directed to methods wherein said adhesion molecule associated with inflammation is ICAM-1.

Preferred embodiments are directed to methods wherein said adhesion molecule associated with inflammation is VLA-4.

Preferred embodiments are directed to methods wherein said adhesion molecule associated with inflammation is Cadherin.

Preferred embodiments are directed to methods wherein said reduction of procoagulant state is accomplished by acceleration of endothelial healing.

Preferred embodiments are directed to methods wherein said acceleration of endothelial healing is facilitated by mobilization of endothelial progenitor cells.

Preferred embodiments are directed to methods wherein said endothelial progenitor cells express CD31.

Preferred embodiments are directed to methods wherein said endothelial progenitor cells express CD133.

Preferred embodiments are directed to methods wherein said endothelial progenitor cells express CD.

Preferred embodiments are directed to methods wherein the effects of inflammation on inducing a hypercoagulable state are inhibited.

Preferred embodiments are directed to methods wherein said hypercoagulable state is induced by viral infection.

Preferred embodiments are directed to methods wherein said viral infection comprises a member of the coronavirus family.

Preferred embodiments are directed to methods wherein said member of said coronavirus family is SARS-CoV-2.

Preferred embodiments are directed to methods wherein said inflammation is caused by cancer.

### BRIEF DESCRIPTION OF THE DRAWINGS

FIG. 1 is a bar graph showing Pterostilbene reduces inflammation induced tissue factor expression.

FIG. 2 is a bar graph showing Thymoquinone reduces inflammation induced tissue factor expression.

FIG. 3 is a bar graph showing EGCG reduces inflammation induced tissue factor expression.

FIG. 4 is a bar graph showing Sulforaphane reduces inflammation induced tissue factor expression.

FIG. 5 is a bar graph showing QUADRAMUNE™ combination reduces inflammation induced tissue factor expression.

### DETAILED DESCRIPTION OF THE INVENTION

The invention provides the novel use of QuadraMune™, and its individual components, as a means of suppressing inflammation induced pro-coagulation cascades. The invention describes that administration of individual ingredients, and/or combinations results in suppression of tissue factor, as well as upregulation of anti-thrombotic molecules such as thrombomodulin, Protein C, anti-thrombin-III and CD39. In one embodiment of the invention, the preservation of endothelial function is maintained by administration of either the individual ingredients, or the combination described in the invention.

Pterostilbene (trans-3,5-dimethoxy-4-hydroxystilbene) is a natural polyphenolic compound, primarily found in fruits, such as blueberries, grapes, and tree wood. It has been demonstrated to possess potent antioxidant and anti-inflammatory properties. It is a dimethylated analog of resveratrol which is found in blueberries [14], and is believed to be one of the active ingredients in ancient Indian Medicine [15]. The pterostilbene molecule is structurally similar to resveratrol, the antioxidant found in red wine that has comparable anti-inflammatory, and anticarcinogenic properties; however, pterostilbene exhibits increased bioavailability due to the presence of two methoxy groups which cause it to exhibit increased lipophilic and oral absorption [16-20]. In animal studies, pterostilbene was shown to have 80% bioavailability comparedto 20% for resveratrol making it potentially advantageous as a therapeutic agent [16].

We have demonstrated the pterostilbene administered in the form of nanostilbene in cancer patients results in increased NK cell activity, as well as interferon gamma production. Additionally, pterostilbene has shown to inhibit inflammatory cytokines associated with ARDS. For example, studies have demonstratedinhibition of interleukin-1 [21], interleukin-6 [22, 23], interleukin-8 [24], and TNF-alpha [25], by pterostilbene.

COVID-19 has been associated with endothelial activation and coagulopathy. It is interesting to note thatnumerous studies have demonstrated endothelial protective effects of pterostilbene. For example, Zhang et al. investigated the anti-apoptotic effects of pterostilbene in vitro and in vivo in mice. Exposure of human umbilical vein VECs (HUVECs) to oxLDL (200 μg/ml) induced cell shrinkage, chromatin condensation, nuclear fragmentation, and cell apoptosis, but pterostilbene protected against such injuries. In addition, PT injection strongly decreased the number of TUNEL-positive cells in the endothelium of atherosclerotic plaque from apoE(−/−) mice. OxLDL increased reactive oxygen species (ROS) levels, NF-κB activation, p53 accumulation, apoptotic protein levels and caspases-9 and -3 activities and decreased mitochondrial membrane potential (MMP) and cytochrome c release in HUVECs. These alterations were attenuated by pretreatment. Pterostilbene inhibited the expression of lectin-like oxLDL receptor-1 (LOX-1) expression in vitro and in vivo. Cotreatment with PT and siRNA of LOX-1 synergistically reduced oxLDL-induced apoptosis in HUVECs. Overexpression of LOX-1 attenuated the protection by pterostilbene and suppressed the effects of pterostilbene on oxLDL-induced oxidative stress. Pterostilbene may protect HUVECs against oxLDL-induced apoptosis by downregulating LOX-1-mediated activation through a pathway involving oxidative stress, p53, mitochondria, cytochrome c and caspase protease [26]. Endothelial protection by pterostilbene [27, 28], and its analogue resveratrol are well known [29, 30].

First. Taking Kalonji increases the potency of the immune system [31, 32]. Specifically, it has been shown that kalonji activates the natural killer cells of the immune system. Natural killer cells, also called NK cells are the body's first line of protection against viruses. It is well known that patients who have low levels of NK cells are very susceptible to viral infections. Kalonji has been demonstrated to increase NK cell activity. In a study published by Dr. Majdalawieh from the American University of Sharjah, Sharjah, United Arab Emirates [33], it was shown that the aqueous extract of Nigella sativa significantly enhances NK cytotoxic activity. According to the authors, this supports the idea that NK cell activation by Kalonji can protect notonly against viruses, but may also explain why some people report this herb has activity against cancer. It is known that NK cells kill virus infected cells but also kill cancer cells. There are several publications that show that Kalonji has effects against cancer [34-48].

Second. Kalonji suppresses viruses from multiplying. If the virus manages to sneak past the immune system and enters the body, studies have shown that Kalonji, and its active ingredients such as thymoquinone, are able to directly stop viruses, such as coronaviruses and others from multiplying. For example, a study published from University of Gaziantep, in Turkey demonstrated that administration of Kalonji extract to cells infected with coronavirus resulted in suppression of coronavirus multiplication and reduction of pathological protein production [49]. Antiviral activity of Kalonji was demonstrated in other studies, for example, for example, viral hepatitis, and others [50].

Third. Kalonji protects the lungs from pathology. Kalonji was also reported by scholars to possess potent anti-inflammatory effects where its active ingredient thymoquinone suppressed effectively the lipopolysaccharide-induced inflammatory reactions and reduced significantly the concentration of nitric oxide, a marker of inflammation [51]. Moreover, Kalonji has been proven to suppress the pathological processes through blocking the activities of IL-1, IL-6, nuclear factor-κB [52], IL-1 β, cyclooxygenase-1, prostaglandin-E2, prostaglandin-D2 [53], cyclocoxygenase-2, and TNF-α [54] that act as potent inflammatory mediators and were reported to play a major role in the pathogenesis of Coronavirus infection.

Fourth. Kalonji protects against sepsis/too much inflammation. In peer reviewed study from King Saud University, Riyadh, Saudi Arabia, scientists examined two sets of mice (n=12 per group), with parallel control groups, were acutely treated with thymoquinone (ingredient from Kalonji) intraperitoneal injections of 1.0 and 2.0 mg/kg body weight, and were subsequently challenged with endotoxin Gram-negative bacteria (LPS O111:B4). In another set of experiments, thymoquinone was administered at doses of 0.75 and 1.0 mg/kg/day for three consecutive days prior to sepsis induction with live Escherichia coli. Survival of various groups was computed, and renal, hepatic and sepsis markers were quantified. Thymoquinone reduced mortality by 80-90% and improved both renal and hepatic biomarker profiles. The concentrationsof IL-1α with 0.75 mg/kg thymoquinone dose was 310.8±70.93 and 428.3±71.32 pg/ml in the 1 mg/kg group as opposed to controls (1187.0±278.64 pg/ml; P<0.05). Likewise, IL-10 levels decreased significantly with 0.75 mg/kg thymoquinone treatment compared to controls (2885.0±553.98 vs. 5505.2±

333.96 pg/ml; P<0.01). Mice treated with thymoquinone also exhibited relatively lower levels of TNF-α and IL-2 (P values=0.1817 and 0.0851, respectively). This study gives strength to the potential clinical relevance of thymoquinone in sepsis-related morbidity and mortality reduction and suggests that human studies should be performed [55].

Sulforaphane [1-isothiocyanato-4-(methylsulfinyl)-butane], an isothiocyanate, is a chemopreventive photochemical which is a potent inducer of phase II enzyme involved in the detoxification of xenobiotics [56]. Sulforaphane is produced from the hydrolysis of glucoraphanin, the most abundant glucosinolate found in broccoli, and also present in other Brassicaceae [57]. Numerous studies have reported preventionof cancer [58-62], as well as cancer inhibitory properties of sulforaphane [63-68]. Importantly, this led to studies which demonstrated anti-inflammatory effects of this compound.

One of the fundamental features of inflammation is production of TNF-alpha from monocytic lineage cells. Numerous studies have shown that sulforaphane is capable of suppressing this fundamental initiator of inflammation, in part through blocking NF-kappa B translocation. For example, Lin et al. compared the anti-inflammatory effect of sulforaphane on LPS-stimulated inflammation in primary peritoneal macrophages derived from Nrf2 (+/+) and Nrf2 (−/−) mice. Pretreatment with sulforaphane in Nrf2 (+/+) primary peritoneal macrophages potently inhibited LPS-stimulated mRNA expression, protein expression and production of TNF-alpha, IL-1beta, COX-2 and iNOS. HO-1 expression was significantly augmentedin LPS-stimulated Nrf2 (+/+) primary peritoneal macrophages by sulforaphane. Interestingly, the anti-inflammatory effect was attenuated in Nrf2 (−/−) primary peritoneal macrophages. We concluded that SFNexerts its anti-inflammatory activity mainly via activation of Nrf2 in mouse peritoneal macrophages [69]. In a similar study, LPS-challenged macrophages were observed for cytokine production with or without sulforaphane pretreatment. Macrophages were pre-incubated for 6 h with a wide range of concentrations of SFN (0 to 50 μM), and then treated with LPS for 24 h. Nitric oxide (NO) concentration and gene expression of different inflammatory mediators, i.e., interleukin (IL)-6, tumor necrosis factor (TNF)-α, and IL-1β, were measured. sulforaphane neither directly reacted with cytokines, nor with NO. To understand the mechanisms, the authors performed analyses of the expression of regulatory enzyme inducible nitic oxide synthase (iNOS), the transcription factor NF-E2-related factor 2 (Nrf2), and its enzyme heme-oxygenase (HO)-1. The results revealed that LPS increased significantly the expression of inflammatory cytokines and concentration of NO in non-treated cells. sulforaphane was able to prevent the expression of NO and cytokines through regulating inflammatory enzyme iNOS and activation of Nrf2/HO-1 signal transduction pathway [70]. These data are significant because studies have shown both TNF-alpha but also interleukin-6 are involved in pathology of COVID-19 [71-81]. The utilization of sulforaphane as a substitute for anti-IL-6 antibodies would be more economical and potentially without associated toxicity. Other studies have also demonstrated ability of sulforaphane to suppress IL-6 [82-84]. Interestingly, a clinical study was performed in 40 healthy overweight subjects (ClinicalTrials.gov ID NCT 03390855). Treatment phase consisted on the consumption of broccoli sprouts (30 g/day) during 10 weeks and the follow-up phase of 10 weeks of normal diet without consumption of these broccoli sprouts. Anthropometric parameters as body fat mass, body weight, and BMI were determined. Inflammation status was assessed by measuring levels of TNF-α, IL-6, IL-1β and C-reactive protein. IL-6 levels significantly decreased (mean values from 4.76 pg/mL to 2.11 pg/mL with 70 days of broccoli consumption, p<0.001) and during control phase the inflammatory levels were maintained at low grade (mean values from 1.20 pg/mL to 2.66 pg/mL, p<0.001). C-reactive protein significantly decreased as well [85].

An additional potential benefit of sulforaphane is its ability to protect lungs against damage. It is known that the major cause of lethality associated with COVID-19 is acute respiratory distress syndrome (ARDS). It was demonstrated that sulforaphane is effective in the endotoxin model of this condition. In one experiments, BALB/c mice were treated with sulforaphane (50 mg/kg) and 3 days later, ARDS was inducedby the administration of LPS (5 mg/kg). The results revealed that sulforaphane significantly decreased lactate dehydrogenase (LDH) activity (as shown by LDH assay), the wet-to-dry ratio of the lungs and the serum levels of interleukin-6 (IL-6) and tumor necrosis factor-α (TNF-α) (measured by ELISA), as well as nuclear factor-κB protein expression in mice with LPS-induced ARDS. Moreover, treatment with sulforaphane significantly inhibited prostaglandin E2 (PGE2) production, and cyclooxygenase-2 (COX-2), matrix metalloproteinase-9 (MMP-9) protein expression (as shown by western blot analysis), as well as inducible nitric oxide synthase (iNOS) activity in mice with LPS-induced ALI. Lastly, the researchers reported pre-treatment with sulforaphane activated the nuclear factor-E2-related factor 2 (Nrf2)/antioxidant response element (ARE) pathway in the mice with LPS-induced ARDS [86].

EGCG is similar to sulforaphane in that it has been reported to possess cancer preventative properties. This compound has been shown to be one of the top therapeutic ingredients in green tea. It is known from epidemiologic studies that green tea consumption associates with chemoprotective effects against cancer [87-97]. In addition, similarly to sulforaphane, EGCG has been shown to inhibit inflammatory mediators. The first suggestion of this were studies shown suppression of the pro-inflammatory transcription factor NF-kappa B. In a detailed molecular study, EGCG, a potent antitumor agent with anti-inflammatory and antioxidant properties was shown to inhibit nitric oxide (NO) generation as a marker of activated macrophages. Inhibition of NO production was observed when cells were cotreated with EGCG and LPS. iNOS activity in soluble extracts of lipopolysaccharide -activated macrophages treated with EGCG (5 and 10 microM) for 6-24 hr was significantly lower than that in macrophages without EGCG treatment. Western blot, reverse transcription-polymerase chain reaction, and Northern blot analyses demonstrated that significantly reduced 130-kDa protein and 4.5-kb mRNA levels of iNOS were expressed inlipopolysaccharide-activated macrophages with EGCG compared with those without EGCG. Electrophoretic mobility shift assay indicated that EGCG blocked the activation of nuclear factor-kappaB, a transcription factor necessary for iNOS induction. EGCG also blocked disappearance of inhibitor kappaB from cytosolic fraction. These results suggest that EGCG decreases the activity and protein levels of iNOS by reducing the expression of iNOS mRNA and the reduction could occur through prevention ofthe binding of nuclear factor-kappaB to the iNOS promoter [98]. Another study supporting ability of EGCG to suppress NF-kappa B examined a model of atherosclerosis in which exposure of macrophage foam cells to TNF-α results in a downregulation of ABCA1 and a decrease in cholesterol efflux to apoA1, which is attenuated by pretreatment with EGCG. Moreover, rather than activating the Liver X receptor (LXR) pathway, inhibition of the TNF-α-induced nuclear factor-κB (NF-κB) activity is detected with EGCG treatment in cells. In order to inhibit the NF-κB activity, EGCG can promote the dissociation of the nuclear factor E2-related factor 2 (Nrf2)-Kelch-like ECH-associated protein 1 (Keap 1) complex; when the released Nrf2 translocates to the nucleus and activates the transcription of genes containing an ARE element inhibition of NF-κB occurs and Keap1 is separated from the complex to directly interact with IKKβ and thus represses NF-κB function [99].

The anti-inflammatory effects of EGCG can be seen in the ability of this compound to potently inhibit IL-6, the COVID-19 associated cytokine, in a variety of inflammatory settings. For example, in a cardiac infarct model, rats were subjected to myocardial ischemia (30 min) and reperfusion (up to 2 h). Rats were treated with EGCG (10 mg/kg intravenously) or with vehicle at the end of the ischemia period followed by a continuous infusion (EGCG 10 mg/kg/h) during the reperfusion period. In vehicle-treated rats, extensive myocardial injury was associated with tissue neutrophil infiltration as evaluated by myeloperoxidase activity, and elevated levels of plasma creatine phosphokinase. Vehicle-treated rats also demonstrated increased plasma levels of interleukin-6. These events were associated with cytosol degradation of inhibitor kappaB-alpha, activation of IkappaB kinase, phosphorylation of c-Jun, and subsequent activation of nuclear factor-kappaB and activator protein-1 in the infarcted heart. In vivo treatment with EGCG reduced myocardial damage and myeloperoxidase activity. Plasma IL-6 and creatine phosphokinase levels were decreased after EGCG administration. This beneficial effect of EGCG was associated with reduction of nuclear factor-kB and activator protein-1 DNA binding [100]. In an inflammatory model of ulcerative colitis (UC) mice were randomly divided into four groups: Normal control, model (MD), 50 mg/kg/day EGCG treatment and 100 mg/kg/day EGCG treatment. The daily disease activity index (DAI) of the mice was recorded, changes in the organizational structure of the colon were observed and the spleen index (SI)was measured. In addition, levels of interleukin (IL)-6, IL-10, IL-17 and transforming growth factor (TGF)-β1 in the plasma and hypoxia-inducible factor (HIF)-1α and signal transducer and activator of transcription (STAT) 3 protein expression in colon tissues were evaluated. Compared with the MD group, the mice in the two EGCG treatment groups exhibited decreased DAIs and SIs and an attenuation in the colonic tissueerosion. EGCG could reduce the release of IL-6 and IL-17 and regulate the mouse splenic regulatory T-cell (Treg)/T helper 17 cell (Th17) ratio, while increasing the plasma levels of IL-10 and TGF-β1 and decreasing the HIF-1α and STAT3 protein expression in the colon. The experiments confirmed that EGCG treated mice with experimental colitis by inhibiting the release of IL-6 and regulating the body Treg/Th17 balance [101].

In patients with COVID-19, the ARDS associated with fatality resembles septic shock in many aspects, including DIC, fever, vascular leakage, and systemic inflammation. Wheeler et al. induced polymicrobialsepsis in male Sprague-Dawley rats (hemodynamic study) and C57BL6 mice (mortality study) via cecal ligation and double puncture (CL2P). Rodents were treated with either EGCG (10 mg/kg intraperitoneally) or vehicle at 1 and 6 h after CL2P and every 12 h thereafter. In the hemodynamic study, mean arterial blood pressure was monitored for 18 h, and rats were killed at 3, 6, and 18 h after CL2P. In the mortality study, survival was monitored for 72 h after CL2P in mice. In vehicle-treated rodents, CL2P was associated with profound hypotension and greater than 80% mortality rate. Epigallocatechin-3-gallate treatment significantly improved both the hypotension and survival [102].

A subsequent study by Li et al. showed intraperitoneal administration of EGCG protected mice against lethal endotoxemia, and rescued mice from lethal sepsis even when the first dose was given 24 hours aftercecal ligation and puncture. The therapeutic effects were partly attributable to: 1) attenuation of systemic accumulation of proinflammatory mediator (e.g., HMGB1) and surrogate marker (e.g., IL-6 and KC) of lethal sepsis; and 2) suppression of HMGB1-mediated inflammatory responses by preventing clustering of exogenous HMGB1 on macrophage cell surface [103].

Finally, in a lung study mice were treated with EGCG (10 mg/kg) intraperitoneally (ip) 1 h before LPS injection (10 mg/kg, ip). The results showed that EGCG attenuated LPS-induced ARDS as it decreased the changes in blood gases and reduced the histological lesions, wet-to-dry weight ratios, and myeloperoxidase. (MPO) activity. In addition, EGCG significantly decreased the expression of pro-inflammatory cytokines tumor necrosis factor (TNF)-α, interleukin (IL)-1β, and IL-6 in the lung, serum, and bronchoalveolar lavage fluid, and alleviated the expression of TLR-4, MyD88, TRIF, and p-p65 in the lung tissue. In addition, it increased the expression of IκB-α and had no influence on the expression of p65. Collectively, these results demonstrated the protective effects of EGCG against LPS-induced ARDS in mice through its anti-inflammatory effect that may be attributed to the suppression of the activation of TLR 4-dependent NF-κB signaling pathways [104].

### EXAMPLES

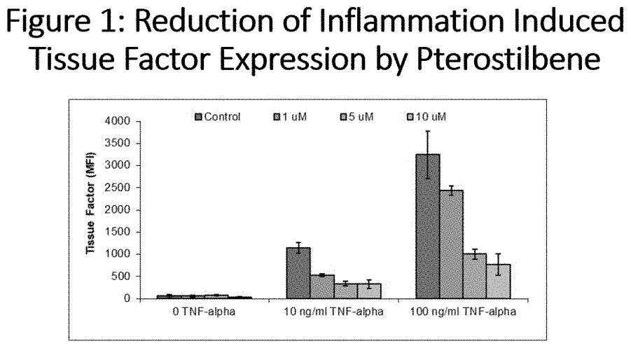

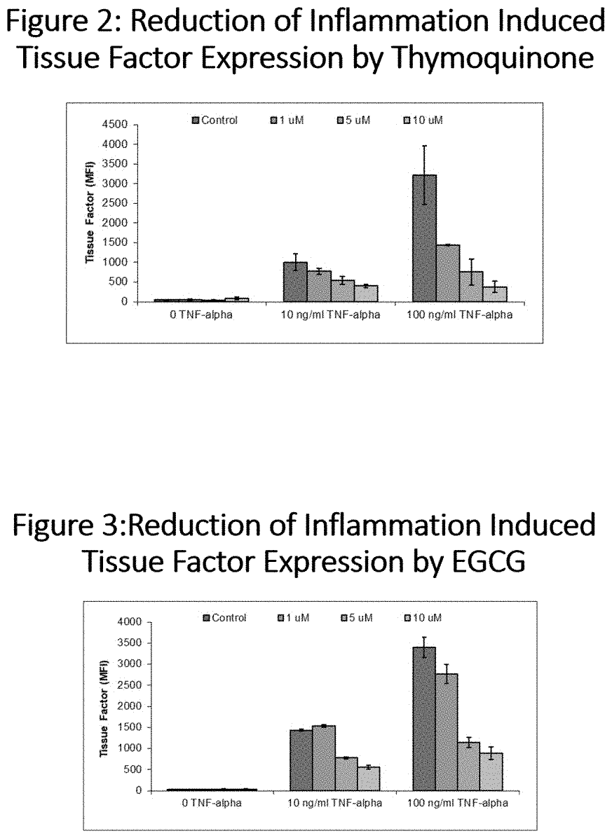

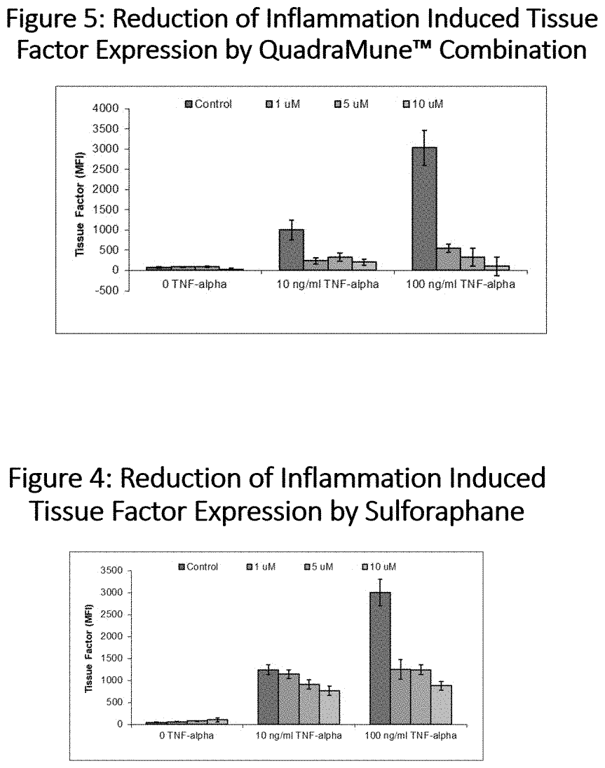

In the experiments below, human umbilical vein endothelial cells (HUVEC) where purchased from AllCells and grown in Opti-MEM media with complete fetal calf serum. Cells were stimulated with the indicated concentrations of TNF-alpha for 48 hours and incubated with the indicated concentrations of individual components of QuadraMune™ as well as the combination. Quantification of Tissue Factor was performedby flow cytometry and expressed as mean fluorescent intensity (MFI). FIG. 1 shows reduction of TNF-alpha induced Tissue Factor expression by pterostilbene. FIG. 2 shows reduction of TNF-alpha induced Tissue Factor expression by thymoquinone. FIG. 3 shows reduction of TNF-alpha induced Tissue Factor expression by sulforaphane. FIG. 4 shows reduction of TNF-alpha induced Tissue Factor expression by EGCG. FIG. 5 shows reduction of TNF-alpha induced Tissue Factor expression by the combination of the four ingredients (QuadraMune™).

### REFERENCES

1. Zhu N, Zhang D, Wang W, Li X, Yang B, Song J, Zhao X, Huang B, Shi W, Lu R et al: A Novel Coronavirus from Patients with Pneumonia in China, 2019. N Engl J Med 2020, 382(8):727-733.
2. Guo Y R, Cao Q D, Hong Z S, Tan Y Y, Chen S D, Jin H J, Tan K S, Wang D Y, Yan Y: The origin, transmission and clinical therapies on coronavirus disease 2019 (COVID-19) outbreak—an update on the status. Mil Med Res 2020, 7(1):11.
3. WHO WHO: Coronavirus disease (COVID-19) outbreak 2020: https://www.who.int/emergencies/diseases/novel-corovirus-2019.
4. Zhang S, Diao M, Yu W, Pei L, Lin Z, Chen D: Estimation of the reproductive number of Novel Coronavirus (COVID-19) and the probable outbreak size on the Diamond Princess cruise ship: A data-driven analysis. Int J Infect Dis 2020.
5. Zhao S, Lin Q, Ran J, Musa S S, Yang G, Wang W, Lou Y, Gao D, Yang L, He D et al: Preliminary estimation of the basic reproduction number of novel coronavirus (2019-nCoV) in China, from 2019 to 2020: A data-driven analysis in the early phase of the outbreak. Int J Infect Dis 2020, 92:214-217.
6. Huang C, Wang Y, Li X, Ren L, Zhao J, Hu Y, Zhang L, Fan G, Xu J, Gu X et al: Clinical features of patients infected with 2019 novel coronavirus in Wuhan, China. Lancet 2020, 395(10223):497-506.
7. Wang D, Hu B, Hu C, Zhu F, Liu X, Zhang J, Wang B, Xiang H, Cheng Z, Xiong Y et al: Clinical Characteristics of 138 Hospitalized Patients With 2019 Novel Coronavirus-Infected Pneumonia in Wuhan, China. JAMA 2020.
8. Chen N, Zhou M, Dong X, Qu J, Gong F, Han Y, Qiu Y, Wang J, Liu Y, Wei Y et al: Epidemiological and clinical characteristics of 99 cases of 2019 novel coronavirus pneumonia in Wuhan, China: a descriptive study. Lancet 2020, 395(10223):507-513.
9. Grasselli G, Pesenti A, Cecconi M: Critical Care Utilization for the COVID-19 Outbreak in Lombardy, Italy: Early Experience and Forecast During an Emergency Response. JAMA 2020.
10. Wu C, Chen X, Cai Y, Xia J, Zhou X, Xu S, Huang H, Zhang L, Zhou X, Du C et al: Risk Factors Associated With Acute Respiratory Distress Syndrome and Death in Patients With Coronavirus Disease 2019 Pneumonia in Wuhan, China. JAMA Intern Med 2020.
11. Shi H, Han X, Jiang N, Cao Y, Alwalid O, Gu J, Fan Y, Zheng C: Radiological findings from 81 patients with COVID-19 pneumonia in Wuhan, China: a descriptive study. Lancet Infect Dis 2020.
12. Xu Z, Shi L, Wang Y, Zhang J, Huang L, Zhang C, Liu S, Zhao P, Liu H, Zhu L et al: Pathological findings of COVID-19 associated with acute respiratory distress syndrome. Lancet Respir Med 2020.
13. Tian S, Hu W, Niu L, Liu H, Xu H, Xiao S Y: Pulmonary pathology of early phase 2019 novel coronavirus (COVID-19) pneumonia in two patients with lung cancer. J Thorac Oncol 2020.
14. McCormack D, McFadden D: A review of pterostilbene antioxidant activity and disease modification. Oxid Med Cell Longev 2013, 2013:575482.
15. Paul B, Masih I, Deopujari J, Charpentier C: Occurrence of resveratrol and pterostilbene in age-old darakchasava, an ayurvedic medicine from India. J Ethnopharmacol 1999, 68(1-3):71-76.
16. Kapetanovic I M, Muzzio M, Huang Z, Thompson T N, McCormick D L: Pharmacokinetics, oral bioavailability, and metabolic profile of resveratrol and its dimethylether analog, pterostilbene, in rats. Cancer Chemother Pharmacol 2011, 68(3):593-601.
17. Perecko T, Drabikova K, Rackova L, Ciz M, Podborska M, Lojek A, Harmatha J, Smidrkal J, Nosal R, Jancinova V: Molecular targets of the natural antioxidant pterostilbene: effect on protein kinase C, caspase-3 and apoptosis in human neutrophils in vitro. Neuro Endocrinol Lett 2010, 31 Suppl 2:84-90.
18. Stivala L A, Savio M, Carafoli F, Perucca P, Bianchi L, Maga G, Forti L, Pagnoni U M, Albini A, Prosperi E et al: Specific structural determinants are responsible for the antioxidant activity and the cell cycle effects of resveratrol. J Biol Chem 2001, 276(25):22586-22594.
19. Athar M, Back J H, Tang X, Kim K H, Kopelovich L, Bickers D R, Kim A L: Resveratrol: a review of preclinical studies for human cancer prevention. Toxicol Appl Pharmacol 2007, 224(3):274-283.
20. Bishayee A: Cancer prevention and treatment with resveratrol: from rodent studies to clinical trials. Cancer Prev Res (Phila) 2009, 2(5):409-418.
21. Hsu C L, Lin Y J, Ho C T, Yen G C: The inhibitory effect of pterostilbene on inflammatory responses during the interaction of 3T3-L1 adipocytes and RAW 264.7 macrophages. J Agric Food Chem 2013, 61(3):602-610.
22. McCormack D, McDonald D, McFadden D: Pterostilbene ameliorates tumor necrosis factor alpha-induced pancreatitis in vitro. J Surg Res 2012, 178(1):28-32.
23. Erasalo H, Hamalainen M, Leppanen T, Maki-Opas I, Laavola M, Haavikko R, Yli-Kauhaluoma J, Moilanen E: Natural Stilbenoids Have Anti-Inflammatory Properties in Vivo and Down-Regulate the Production of Inflammatory Mediators NO, IL6, and MCPJ Possibly in a PI3K/Akt-Dependent Manner. J Nat Prod 2018, 81(5):1131-1142.
24. Allijn I E, Vaessen S F, Quarles van Ufford L C, Beukelman K J, de Winther M P, Storm G, Schiffelers R M: Head-to-Head Comparison of Anti-Inflammatory Performance of Known Natural Products In Vitro. PLoS One 2016, 11(5):e0155325.
25. Meng X L, Yang J Y, Chen G L, Wang L H, Zhang U, Wang S, Li J, Wu C F: Effects of resveratrol and its derivatives on lipopolysaccharide-induced microglial activation and their structure-activity relationships. Chem Biol Interact 2008, 174(1):51-59.
26. Zhang L, Zhou G, Song W, Tan X, Guo Y, Zhou B, Jing H, Zhao S, Chen L: Pterostilbene protects vascular endothelial cells against oxidized low-density lipoprotein-induced apoptosis in vitro and in vivo. Apoptosis 2012, 17(1):25-36.
27. Park S H, Jeong S O, Chung H T, Pae H O: Pterostilbene, an Active Constituent of Blueberries, Stimulates Nitric Oxide Production via Activation of Endothelial Nitric Oxide Synthase in Human Umbilical Vein Endothelial Cells. Plant Foods Hum Nutr 2015, 70(3):263-268.
28. Chen Z W, Miu H F, Wang H P, Wu Z N, Wang W J, Ling Y J, Xu X H, Sun H J, Jiang X: Pterostilbene protects against uraemia serum-induced endothelial cell damage via activation of Keap1/Nrf2/HO-1 signaling. Int Urol Nephrol 2018, 50(3):559-570.
29. Chen C, Song C, Zhang D, Yin D, Zhang R, Chen J, Dou K: Effect of resveratrol combined with atorvastatin on re-endothelialization after drug-eluting stents implantation and the underlying mechanism. Life Sci 2020, 245:117349.
30. Bekpinar S, Karaca E, Yamakoglu S, Alp-Yildirim F I, Olgac V, Uydes-Dogan B S, Cibali E, Gultepe S, Uysal M: Resveratrol ameliorates the cyclosporine-induced vascular and renal impairments: possible impact of the modulation of renin-angiotensin system. Can J Physiol Pharmacol 2019, 97(12):1115-1123.
31. Swamy S M, Tan B K: Cytotoxic and immunopotentiating effects of ethanolic extract of Nigella sativa L. seeds. J Ethnopharmacol 2000, 70(1):1-7.
32. Salem M L, Alenzi F Q, Attia W Y: Thymoquinone, the active ingredient of Nigella sativa seeds, enhances survival and activity of antigen-specific CD8-positive T cells in vitro. Br J Biomed Sci 2011, 68(3):131-137.
33. Majdalawieh A F, Hmaidan R, Can R I: Nigella sativa modulates splenocyte proliferation, Th1/Th2 cytokine profile, macrophage function and NK anti-tumor activity. J Ethnopharmacol 2010, 131(2):268-275.
34. Salomi M J, Panikkar K R, Kesavan M, Donata K, Sr., Rajagopalan K: Anti-cancer activity of nigella sativa. Anc Sci Lift 1989, 8(3-4):262-266.
35. Salomi N J, Nair S C, Jayawardhanan K K, Varghese C D, Panikkar K R: Antitumour principles from Nigella sativa seeds. Cancer Lett 1992, 63(1):41-46.
36. Ait Mbarek L, Ait Mouse H, Elabbadi N, Bensalah M, Gamouh A, Aboufatima R, Benharref A, Chait A, Kamal M, Dalal A et al: Anti-tumor properties of blackseed (Nigella sativa L.) extracts. Braz J Med Biol Res 2007, 40(6):839-847.
37. Amara A A, El-Masry M H, Bogdady H H: Plant crude extracts could be the solution: extracts showing in vivo antitumorigenic activity. Pak J Pharm Sci 2008, 21(2):159-171.
38. Banerjee S, Padhye S, Azmi A, Wang Z, Philip P A, Kucuk O, Sarkar F H, Mohammad R M: Review on molecular and therapeutic potential of thymoquinone in cancer. Nutr Cancer 2010, 62(7):938-946.
39. Khan M A, Chen H C, Tania M, Zhang D Z: Anticancer activities of Nigella sativa (black cumin). Afr J Tradit Complement Altern Med 2011, 8(5 Suppl):226-232.
40. Woo C C, Kumar A P, Sethi G, Tan K H: Thymoquinone: potential cure for inflammatory disorders and cancer. Biochem Pharmacol 2012, 83(4):443-451.
41. Lei X, Lv X, Liu M, Yang Z, Ji M, Guo X, Dong W: Thymoquinone inhibits growth and augments 5-fluorouracil-induced apoptosis in gastric cancer cells both in vitro and in vivo. Biochem Biophys Res Commun 2012, 417(2):864-868.
42. Linjawi S A, Khalil W K, Hassanane M M, Ahmed E S: Evaluation of the protective effect of Nigella sativa extract and its primary active component thymoquinone against DMBA-induced breast cancer in female rats. Arch Med Sci 2015,11(1):220-229.
43. Majdalawieh A F, Fayyad M W: Recent advances on the anti-cancer properties of Nigella sativa, a widely used food additive. J Ayurveda Integr Med 2016, 7(3):173-180.
44. Majdalawieh A F, Fayyad M W, Nasrallah G K: Anti-cancer properties and mechanisms of action of thymoquinone, the major active ingredient of Nigella sativa. Crit Rev Food Sci Nutr 2017, 57(18):3911-3928.
45. Mostofa A G M, Hossain M K, Basak D, Bin Sayeed M S: Thymoquinone as a Potential Adjuvant Therapy for Cancer Treatment: Evidence from Preclinical Studies. Front Pharmacol 2017, 8:295.
46. Asaduzzaman Khan M, Tania M, Fu S, Fu J: Thymoquinone, as an anticancer molecule: from basic research to clinical investigation. Oncotarget 2017, 8(31):51907-51919.
47. Imran M, Rauf A, Khan I A, Shahbaz M, Qaisrani T B, Fatmawati S, Abu-Izneid T, Imran A, Rahman K U, Gondal T A: Thymoquinone: A novel strategy to combat cancer: A review. Biomed Pharmacother 2018, 106:390-402.
48. Zhang Y, Fan Y, Huang S, Wang G, Han R, Lei F, Luo A, Jing X, Zhao L, Gu S et al: Thymoquinone inhibits the metastasis of renal cell cancer cells by inducing autophagy via AMPK/mTOR signaling pathway. Cancer Sci 2018, 109(12):3865-3873.
49. Ulasli M, Gurses S A, Bayraktar R, Yumrutas O, Oztuzcu S, Igci M, Igci Y Z, Cakmak E A, Arslan A: The effects of Nigella sativa (Ns), Anthemis hyalina (Ah) and Citrus sinensis (Cs) extracts on the replication of coronavirus and the expression of TRP genes family. Mol Biol Rep 2014, 41(3):1703-1711.
50. Ahmad A, Husain A, Mujeeb M, Khan S A, Najmi A K, Siddique N A, Damanhouri Z A, Anwar F: A review on therapeutic potential of Nigella sativa: A miracle herb. Asian Pac J Trop Biomed 2013, 3(5):337-352.
51. Alemi M, Sabouni F, Sanjarian F, Haghbeen K, Ansari S: Anti-inflammatory effect of seeds and callus of Nigella sativa L. extracts on mix glial cells with regard to their thymoquinone content. AAPS PharmSciTech 2013, 14(1):160-167.
52. Shuid A N, Mohamed N, Mohamed I N, Othman F, Suhaimi F, Mohd Ramli E S, Muhammad N, Soelaiman I N: Nigella sativa: A Potential Antiosteoporotic Agent. Evid Based Complement Alternat Med 2012, 2012:696230.
53. El Mezayen R, El Gazzar M, Nicolls M R, Marecki J C, Dreskin S C, Nomiyama H: Effect of thymoquinone on cyclooxygenase expression and prostaglandin production in a mouse model of allergic airway inflammation. Immunol Lett 2006, 106(1):72-81.
54. Chehl N, Chipitsyna G, Gong Q, Yeo C J, Arafat HA: Anti-inflammatory effects of the Nigella sativa seed extract, thymoquinone, in pancreatic cancer cells. HPB (Oxford) 2009, 11(5):373-381.
55. Alkharfy K M, Al-Daghri N M, Al-Attas O S, Alokail M S: The protective effect of thymoquinone against sepsis syndrome morbidity and mortality in mice. Int Immunopharmacol 2011, 11(2):250-254.
56. Shen G, Khor T O, Hu R, Yu S, Nair S, Ho C T, Reddy B S, Huang M T, Newmark H L, Kong A N: Chemoprevention of familial adenomatous polyposis by natural dietary compounds sulforaphane and dibenzoylmethane alone and in combination in ApcMin/+ mouse. Cancer Res 2007, 67(20):9937-9944.
57. Zambrano V, Bustos R, Mahn A: Insights about stabilization of sulforaphane through microencapsulation. Heliyon 2019, 5(11):e02951.
58. Steinkellner H, Rabot S, Freywald C, Nobis E, Scharf G, Chabicovsky M, Knasmuller S, Kassie F: Effects of cruciferous vegetables and their constituents on drug metabolizing enzymes involved in the bioactivation of DNA-reactive dietary carcinogens. Mutat Res 2001, 480-481:285-297.
59. Fahey J W, Zhang Y, Talalay P: Broccoli sprouts: an exceptionally rich source of inducers of enzymes that protect against chemical carcinogens. Proc Natl Acad Sci USA 1997, 94(19):10367-10372.
60. Solowiej E, Kasprzycka-Guttman T, Fiedor P, Rowinski W: Chemoprevention of cancerogenesis—the role of sulforaphane. Acta Pol Pharm 2003, 60(1):97-100.
61. Gills J J, Jeffery E H, Matusheski N V, Moon R C, Lantvit D D, Pezzuto J M: Sulforaphane prevents mouse skin tumorigenesis during the stage of promotion. Cancer Lett 2006, 236(1):72-79.
62. Myzak M C, Dashwood W M, Orner G A, Ho E, Dashwood R H: Sulforaphane inhibits histone deacetylase in vivo and suppresses tumorigenesis in Apc-minus mice. FASEB J 2006, 20(3):506-508.
63. Singh A V, Xiao D, Lew K L, Dhir R, Singh S V: Sulforaphane induces caspase-mediated apoptosis in cultured PC-3 human prostate cancer cells and retards growth of PC-3 xenografts in vivo. Carcinogenesis 2004, 25(1):83-90.
64. Wang L, Liu D, Ahmed T, Chung F L, Conaway C, Chiao J W: Targeting cell cycle machinery as a molecular mechanism of sulforaphane in prostate cancer prevention. Int J Oncol 2004, 24(1):187-192.
65. Pham N A, Jacobberger J W, Schimmer A D, Cao P, Gronda M, Hedley D W: The dietary isothiocyanate sulforaphane targets pathways of apoptosis, cell cycle arrest, and oxidative stress in human pancreatic cancer cells and inhibits tumor growth in severe combined immunodeficient mice. Mol Cancer Ther 2004, 3(10):1239-1248.
66. Thejass P, Kuttan G: Antimetastatic activity of Sulforaphane. Life Sci 2006, 78(26):3043-3050.
67. Fimognari C, Hrelia P: Sulforaphane as a promising molecule for fighting cancer. Mutat Res 2007, 635(2-3):90-104.
68. Li Y, Zhang T, Korkaya H, Liu S, Lee HF , Newman B, Yu Y, Clouthier S G, Schwartz S J, Wicha M S et al: Sulforaphane, a dietary component of broccoli/broccoli sprouts, inhibits breast cancer stem cells. Clin Cancer Res 2010, 16(9):2580-2590.
69. Lin W, Wu R T, Wu T, Khor T O, Wang H, Kong A N: Sulforaphane suppressed LPS-induced inflammation in mouse peritoneal macrophages through Nrf2 dependent pathway. Biochem Pharmacol 2008, 76(8):967-973.
70. Ruhee R T, Ma S, Suzuki K: Sulforaphane Protects Cells against Lipopolysaccharide-Stimulated Inflammation in Murine Macrophages. Antioxidants (Basel) 2019, 8(12).
71. Xu X, Han M, Li T, Sun W, Wang D, Fu B, Zhou Y, Zheng X, Yang Y, Li X et al: Effective treatment of severe COVID-19 patients with tocilizumab. Proc Natl Acad Sci USA 2020.
72. Liu F, Li L, Xu M, Wu J, Luo D, Zhu Y, Li B, Song X, Zhou X: Prognostic value of interleukin-6, C-reactive protein, and procalcitonin in patients with COVID-19. J Clin Virol 2020, 127:104370.
73. Aziz M, Fatima R, Assaly R: Elevated Interleukin-6 and Severe COVID-19: A Meta-Analysis. J Med Virol 2020.
74. Chen X, Zhao B, Qu Y, Chen Y, Xiong J, Feng Y, Men D, Huang Q, Liu Y, Yang B et al: Detectable serum SARS-CoV-2 viral load (RNAaemia) is closely correlated with drastically elevated interleukin 6 (IL-6) level in critically ill COVID-19 patients. Clin Infect Dis 2020.
75. Zhang C, Wu Z, Li J W, Zhao H, Wang G Q: The cytokine release syndrome (CRS) of severe COVID-19 and Interleukin-6 receptor (IL-6R) antagonist Tocilizumab may be the key to reduce the mortality. Int J Antimicrob Agents 2020:105954.
76. Zhang X, Song K, Tong F, Fei M, Guo H, Lu Z, Wang J, Zheng C: First case of COVID-19 in a patient with multiple myeloma successfully treated with tocilizumab. Blood Adv 2020, 4(7): 1307-1310.
77. McGonagle D, Sharif K, O'Regan A, Bridgewood C: The Role of Cytokines including Interleukin-6 in COVID-19 induced Pneumonia and Macrophage Activation Syndrome-Like Disease. Autoimmun Rev 2020:102537.
78. Luo P, Liu Y, Qiu L, Liu X, Liu D, Li J: Tocilizumab treatment in COVID-19: A single center experience. J Med Virol 2020.
79. Ulhaq Z S, Soraya G V: Interleukin-6 as a potential biomarker of COVID-19 progression. Med Mal Infect 2020.
80. Fu B, Xu X, Wei H: Why tocilizumab could be an effective treatment for severe COVID-19? J Transl Med 2020, 18(1):164.
81. Liu B, Li M, Zhou Z, Guan X, Xiang Y: Can we use interleukin-6 (IL-6) blockade for coronavirus disease 2019 (COVID-19)-induced cytokine release syndrome (CRS)? J Autoimmun 2020:102452.
82. Eren E, Tufekci K U, Isci K B, Tastan B, Genc K, Genc S: Sulforaphane Inhibits Lipopolysaccharide-Induced Inflammation, Cytotoxicity, Oxidative Stress, and miR-155 Expression and Switches to Mox Phenotype through Activating Extracellular Signal-Regulated Kinase 1/2-Nuclear Factor Erythroid 2-Related Factor 2/Antioxidant Response Element Pathway in Murine Microglial Cells. Front Immunol 2018, 9:36.
83. Ma T, Zhu D, Chen D, Zhang Q, Dong H, Wu W, Lu H, Wu G: Sulforaphane, a Natural Isothiocyanate Compound, Improves Cardiac Function and Remodeling by Inhibiting Oxidative Stress and Inflammation in a Rabbit Model of Chronic Heart Failure. Med Sci Monit 2018, 24:1473-1483.
84. Liu H, Zimmerman A W, Singh K, Connors S L, Diggins E, Stephenson K K, Dinkova-Kostova A T, Fahey J W: Biomarker Exploration in Human Peripheral Blood Mononuclear Cells for Monitoring Sulforaphane Treatment Responses in Autism Spectrum Disorder. Sci Rep 2020, 10(1):5822.
85. Lopez-Chillon M T, Carazo-Diaz C, Prieto-Merino D, Zafrilla P, Moreno D A, Villano D: Effects of long-term consumption of broccoli sprouts on inflammatory markers in overweight subjects. Clin Nutr 2019, 38(2):745-752.
86. Qi T, Xu F, Yan X, Li S, Li H: Sulforaphane exerts anti-inflammatory effects against lipopolysaccharide-induced acute lung injury in mice through the Nrf2/ARE pathway. Int J Mol Med 2016, 37(1):182-188.
87. Dashwood R H, Xu M, Hernaez J F, Hasaniya N, Youn K, Razzuk A: Cancer chemopreventive mechanisms of tea against heterocyclic amine mutagens from cooked meat. Proc Soc Exp Biol Med 1999, 220(4):239-243.
88. Brown M D: Green tea (Camellia sinensis) extract and its possible role in the prevention of cancer. Altern Med Rev 1999, 4(5):360-370.
89. Banerjee S, Manna S, Mukherjee S, Pal D, Panda C K, Das S: Black tea polyphenols restrict benzopyrene-induced mouse lung cancer progression through inhibition of Cox-2 and induction of caspase-3 expression. Asian Pac J Cancer Prev 2006, 7(4):661-666.
90. Shimizu M, Shirakami Y, Moriwaki H: Targeting receptor tyrosine kinases for chemoprevention by green tea catechin, EGCG. Int J Mol Sci 2008, 9(6):1034-1049.
91. Johnson J J, Bailey R H, Mukhtar H: Green tea polyphenols for prostate cancer chemoprevention: a translational perspective. Phytomedicine 2010,17(1):3-13.
92. Kim J W, Amin A R, Shin D M: Chemoprevention of head and neck cancer with green tea polyphenols. Cancer Prev Res (Phila) 2010, 3(8):900-909.
93. Henning S M, Wang P, Heber D: Chemopreventive effects of tea in prostate cancer: green tea versus black tea. Mol Nutr Food Res 2011, 55(6):905-920.
94. Du G J, Zhang Z, Wen X D, Yu C, Calway T, Yuan C S, Wang C Z: Epigallocatechin Gallate (EGCG) is the most effective cancer chemopreventive polyphenol in green tea. Nutrients 2012, 4(11):1679-1691.
95. Henning S M, Wang P, Abgaryan N, Vicinanza R, de Oliveira D M, Zhang Y, Lee R P, Carpenter C L, Aronson W J, Heber D: Phenolic acid concentrations in plasma and urine from men consuming green or black tea and potential chemopreventive properties for colon cancer. Mol Nutr Food Res 2013, 57(3):483-493.
96. Schramm L: Going Green: The Role of the Green Tea Component EGCG in Chemoprevention. J Carcinog Mutagen 2013, 4(142):1000142.
97. Rahmani A H, Al Shabrmi F M, Allemailem K S, Aly S M, Khan M A: Implications of Green Tea and Its Constituents in the Prevention of Cancer via the Modulation of Cell Signalling Pathway. Biomed Res Int 2015, 2015:925640.
98. Lin Y L, Lin J K: (−)-Epigallocatechin-3-gallate blocks the induction of nitric oxide synthase by down-regulating lipopolysaccharide-induced activity of transcription factor nuclear factor-kappaB. Mol Pharmacol 1997, 52(3):465-472.
99. Jiang J, Mo Z C, Yin K, Zhao G J, Lv Y C, Ouyang X P, Jiang Z S, Fu Y, Tang C K: Epigallocatechin-3-gallate prevents TNF-alpha-induced NF-kappaB activation thereby upregulating ABCA1 via the Nrf2/Keap1 pathway in macrophage foam cells. Int J Mol Med 2012, 29(5):946-956.
100. Aneja R, Hake P W, Burroughs T J, Denenberg A G, Wong H R, Zingarelli B: Epigallocatechin, a green tea polyphenol, attenuates myocardial ischemia reperfusion injury in rats. Mol Med 2004, 10(1-6):55-62.
101. Xu Z, Wei C, Zhang R U, Yao J, Zhang D, Wang L: Epigallocatechin-3-gallate-induced inhibition of interleukin-6 release and adjustment of the regulatory T/T helper 17 cell balance in the treatment of colitis in mice. Exp Ther Med 2015, 10(6):2231-2238.
102. Wheeler D S, Lahni P M, Hake P W, Denenberg A G, Wong H R, Snead C, Catravas J D, Zingarelli B: The green tea polyphenol epigallocatechin-3-gallate improves systemic hemodynamics and survival in rodent models of polymicrobial sepsis. Shock 2007, 28(3):353-359.
103. Li W, Ashok M, Li J, Yang H, Sama A E, Wang H: A major ingredient of green tea rescues mice from lethal sepsis partly by inhibiting HMGB1. PLoS One 2007, 2(11):e1153.
104. Wang J, Fan S M, Zhang J: Epigallocatechin-3-gallate ameliorates lipopolysaccharide-inducedacute lung injury by suppression of TLR4/NF-kappaB signaling activation. Braz J Med Biol Res 2019, 52(7):e8092.

1. A method of reducing inflammation associated hypercoagulation states comprising administration of a therapeutic combination comprising: a) Green Tea and/or extract thereof; b) Blueberry and/or extract thereof; c) Nigella Sativa and/or extract thereof; and d) broccoli and/or extract thereof.

2. The method of claim 1, wherein said green tea extract is epigallocatechin-3-gallate or an analogue thereof.

3. The method of claim 1, wherein said blueberry extract is pterostilbene or an analogue thereof.

4. The method of claim 1, wherein said Nigella Sativa extract is thymoquinone or an analogue thereof.

5. The method of claim 1, wherein said broccoli extract is sulforaphane or an analogue thereof.

6. The method of claim 1, wherein said therapeutic combination is administered at a dosage and frequency sufficient to inhibit tissue factor expression.

7. The method of claim 6, wherein said tissue factor expression is on the endothelium.

8. The method of claim 6, wherein said tissue factor expression is on microglia.

9. The method of claim 6, wherein said tissue factor expression is on the monocytes.

10. The method of claim 6, wherein said tissue factor expression is on pulmonary endothelium.

11. The method of claim 6, wherein said tissue factor expression is on the renal endothelium.

12. The method of claim 1, wherein said therapeutic combination is Quadramune™.

13. The method of claim 12, wherein said QuadraMune is administered at a concentration of 10 mg to 10 grams per day.

14. The method of claim 12, wherein said QuadraMune is administered at a concentration of 100 mg to 2 grams per day.

15. The method of claim 12, wherein said QuadraMune is administered at a concentration of 200 mg to 1 gram per day.

17. The method of claim 1, wherein said hypercoagulation state is caused by viral infection.

18. The method of claim 1, wherein said therapeutic mixture decreases hypercoagulability state by inducing upregulated expression of thrombomodulin.

19. The method of claim 1, wherein said therapeutic mixture decreases hypercoagulability state by inducing upregulated expression of anti-thrombin III.

20. The method of claim 1, wherein said therapeutic mixture decreases hypercoagulability state by inducing upregulated expression of Protein C
